(19) United States
(12) Patent Application Publication
(10) Pub. No.: US 2021/0290508 A1
(43) Pub. Date: Sep. 23, 2021

(54) SYSTEMS, METHODS AND COMPOSITIONS FOR EFFECTIVE SANITIZING OR DISINFECTING

(71) Applicant: Soulshine Development Group, Inc., Seattle, WA (US)

(72) Inventors: Patrick J. Wlaznak, Seattle, WA (US); Robert William LaChance, II, Seattle, WA (US)

(73) Assignee: Soulshine Development Group, Inc., Seattle, WA (US)

(21) Appl. No.: 17/209,293

(22) Filed: Mar. 23, 2021

Related U.S. Application Data
(60) Provisional application No. 62/993,086, filed on Mar. 23, 2020.

(51) Int. Cl. A61K 8/34; B01J 19/18; B01J 19/00; B01J 14/00; A61Q 17/00; A61K 8/9794

(52) U.S. Cl. CPC A61K 8/347; B01J 19/1862; B01J 19/0066; B01J 14/00; A61Q 17/005; A61K 2800/805; A61K 8/9794; B01J 2219/00481; A61K 2800/48; A61K 2800/70; A61K 8/345

(57) ABSTRACT

Apparatus and methods for producing a sanitizing composition are described. The apparatus includes: (1) one or more first reactors for heating alcohol with a cannabidiol (“CBD”) and/or a cannabigerol (CBG) to produce an intermediate sanitizing composition; and (2) a first homogenizing subsystem including a first homogenizing blade for mixing alcohol with the intermediate sanitizing composition to produce a first homogenized composition. Methods of the present teachings include: (1) mixing alcohol with a CBD and/or a CBG to produce an intermediate sanitizing composition; (2) cooling the intermediate sanitizing composition to a temperature that ranges from about 10° C. to about 30° C. to produce a cooled intermediate sanitizing composition; and (3) admixing the cooled intermediate sanitizing composition with an alcohol and water solution to produce the sanitizing composition.

### RELATED APPLICATION

The application claims priority from U.S. Provisional Application having Serial No. 62/993,086 filed on Mar. 23, 2020, which is incorporated herein by reference for all purposes.

### FIELD

The present teachings generally relate to systems, methods and compositions for effectively sanitizing or disinfecting against viruses and bacteria. More particularly, the present teachings relate to systems, methods and compositions, which use effective amounts of cannabidiol (“CBD”) and/or cannabigerol (CBG), for effectively sanitizing or disinfecting against viruses and bacteria.

### BACKGROUND

Aside from washing hands, hand sanitizers are commonly used as a protective measure to sanitize against viral or bacterial attacks. During a virus outbreak (e.g., a virus pandemic), for example, hand sanitizers are more frequently used to combat the spreading virus contagion. However, although typical hand-sanitizer compositions attempt to effectively sanitize, they are essentially skin-irritants and their frequent use causes skin conditions that are painful. Moreover, hands and fingers may be inflamed, due to frequent use, to such an extent that users are debilitated in carrying out their daily activities.

What are, therefore, needed are novel systems, methods and compositions that relate to effective sanitizing and/or disinfecting compositions for sanitizing or disinfecting against contagions, without suffering from the drawbacks encountered when using conventional compositions (e.g., causing painful skin irritations).

### SUMMARY

To achieve the foregoing, the present teachings provide novel systems and methods that produce sanitizing and disinfecting compositions for effectively sanitizing and disinfecting against contagions, without suffering from the drawbacks of such conventional compositions, such as painful skin irritations caused by conventional sanitizing compositions. The present teachings also provide such ultimately produced novel sanitizing or disinfecting compositions for rendering many different types of surfaces free of contagions.

In one aspect, the present arrangements provide different types of apparatus for producing a sanitizing composition. One such exemplar apparatus comprises one or more first reactors and a first homogenizing subsystem. One or more first reactors are designed to heat alcohol (e.g., ethanol) in the presence of a cannabidiol (“CBD”) and/or a cannabigerol (CBG) to produce an intermediate sanitizing composition. In the intermediate sanitizing composition, the alcohol is present at a temperature that is between about 50° C. and about 70° C. To maintain such temperatures, one or more of the first reactors are, preferably, surrounded by a heating jacket. The first homogenizing subsystem includes a first homogenizing blade for mixing alcohol with the intermediate sanitizing composition to produce a first homogenized composition or, in the alternate, an intermediate sanitizing composition. To facilitate discussion, the term “intermediate sanitizing composition,” as used in this specification, also refers to the “first homogenized composition” that may result from a multi-stage reaction system.

In one preferred embodiment, the present arrangements further comprise a first recirculation line that recirculates an output (e.g., the first homogenized composition) residing in one or more of the first reactors from and back to one or more of the first reactors. In this recirculating configuration, the first recirculation line has disposed thereon or proximate thereto a first homogenizing subsystem for homogenizing the output from one or more of the first reactors.

The first homogenizing subsystem is, preferably, located downstream from one or more of the first reactors. The first homogenizing subsystem may further comprise a first motor, a first rotor, a first shaft, a first axle and a first homogenizing blade. In an assembled state of the first homogenizing subsystem, the first motor is coupled to rotationally drive the first axle, which is coupled to the first rotor. Further, the first axle longitudinally extends inside the first shaft. In this subsystem, the first rotor is received within a central region of the first homogenizing blade, defining a first gap therebetween.

During an operative state of the first homogenizing subsystem, the first rotor rotates, based upon the powered first motor, relative to the first homogenizing blade, which may not be rotating. In this operative state, the first homogenizing subsystem not only homogenizes the fluid that flows therethrough (i.e., the first gap between the first rotor and the first homogenizing blade), but also behaves like a pump, i.e., pumps the intermediate sanitizing composition flowing out of the first reactor through the first recirculating line and back to one or more of the first reactors. Moreover, the first blade has first homogenizing teeth that protrude, from a first teeth base portion, a protruding distance, preferably, ranging from about 0.5 mm to about 1.2 mm to facilitate homogenization of the intermediate sanitizing composition having a high viscosity ranging from about 1.05 centipoise (“cp”) to about 1.2 cp.

The present arrangements further comprise one or more valves (e.g., a single three-way valve) designed to direct fluid inside one or more of the first reactors to the first recirculation line or to a second reaction stage.

Preferred embodiments of the present arrangements further comprise one or more second reactors and a second homogenizing subsystem. One or more of the second reactors are designed for reacting and mixing the intermediate sanitizing composition and an alcohol and water solution to produce the sanitizing composition. The second homogenizing subsystem, like the first homogenizing subsystem, facilitates in homogenizing a composition, such as the second homogenized composition or the sanitizing composition of the present teachings. Along this line of structural features, the present arrangements, preferably, further comprise a second recirculation line that recirculates an output (e.g., the second homogenized composition or the sanitizing composition) residing in one or more of the second reactors, from and back to one or more of the second reactors. The second recirculation line has disposed thereon or proximate thereto the second homogenizing subsystem for homogenizing the output from the one or more of the second reactors.

To this end, the second homogenizing subsystem is, preferably, located downstream from one or more of second reactors and further comprises a second motor, a second rotor, a second shaft, a second axle and the second homogenizing blade, which are substantially similar to their counterparts in the first homogenizing subsystem. As a result, in an assembled state of the second homogenizing subsystem, the second motor is coupled to rotationally drive the second axle, which is coupled to the second rotor. The second axle longitudinally extends within the second shaft, which may include a bushing to receive the second homogenizing blade. In this configuration, the second rotor is received within a central region of the second homogenizing blade, defining a second gap therebetween.

During an operative state of the second homogenizing subsystem, the fluid that flows between the second gap is effectively homogenized by the action of the rotating second rotor relative to the second homogenizing blade, which is preferably not rotating. The second homogenizing subsystem, like the first homogenizing subsystem, not only homogenizes the fluid flowing therethrough (e.g., the second gap), but also functions like a pump, i.e., pumping the fluid flowing out of one or more second reactors through the second recirculation line. The second homogenizing blade has second homogenizing teeth that protrude, from a second teeth base portion, a protruding distance ranging from about 1.25 mm to about 3.5 mm for facilitating homogenization of the fluid passing through the second gap and pumping the fluid through the second recirculation line.

In another aspect, the present teachings provide methods for producing a sanitizing composition. One such exemplar method for sanitizing comprises: mixing, using a first reactor, alcohol with a CBD and/or a CBG to produce an intermediate sanitizing composition; cooling the intermediate sanitizing composition to produce a cooled intermediate sanitizing composition; and admixing, using a second reactor, the cooled intermediate sanitizing composition with an alcohol and water solution to produce the sanitizing composition. In the intermediate sanitizing composition, the alcohol is present at a temperature that is between about 50° C. and about 70° C., and in a concentration that ranges from about 75% (by volume) to about 95% (by volume). The cooled intermediate sanitizing composition is at a temperature that ranges from about 10° C. to about 30° C. In the sanitizing composition, the alcohol concentration ranges from about 65% (by volume) to about 95% (by volume).

In a preferred embodiment of the mixing step, for one part by volume of the alcohol, each of CBD or CBG are present in about one part by volume to produce the intermediate sanitizing composition. In the event a mixture of CBD and CBG is used in the mixing step, for one part by volume of the alcohol, a mixture of CBD and CBG is, preferably, present in about one part by volume to produce the intermediate sanitizing composition.

Prior to cooling the intermediate sanitizing composition, the present methods may include preparing the intermediate sanitizing composition for thickening by cooling the intermediate sanitizing composition to a temperature that ranges from about 30° C. and to about 50° C. to produce a thickened intermediate sanitizing composition. The preparing step, preferably, includes introducing into the reactor a thickening agent (e.g., glycerin and/or an aloe-based compound).

In one preferred embodiment, the mixing step includes recirculating, using a first recirculation line, the intermediate sanitizing composition from the first reactor back to the first reactor, and homogenizing the intermediate sanitizing composition. According to the present teachings, effective homogenizing is carried out using a first homogenizing blade having a first homogenizing teeth that protrude, from a first teeth base portion, a protruding distance ranging from about 0.5 mm to about 1.2 mm. The first homogenizing blade is, preferably, disposed on or proximate to the first recirculating line. As mentioned above, homogenizing, preferably, includes passing the intermediate sanitizing composition through a first gap defined between a first homogenizing blade, which preferably does not rotate, and a first rotor, which rotates.

In one preferred embodiment, the admixing step, like the mixing step, includes recirculating, using a second recirculation line, the sanitizing composition from the second reactor back to the second reactor, and homogenizing the sanitizing composition. In one embodiment of the present teachings, effective homogenization of the sanitizing composition is carried out using a second homogenizing blade having a second homogenizing teeth that protrude, from a second teeth base portion, a protruding distance ranging from about 1.25 mm to about 3.5 mm. The second homogenizing blade is, preferably, disposed on or proximate to the second recirculating line. Regardless of the location of the second homogenizing blade, homogenizing, preferably, includes passing the intermediate sanitizing composition through a second gap defined between the second homogenizing blade, which preferably does not rotate, and the second rotor, which rotates.

In yet another aspect, the present teachings provide hand sanitizing compositions. One such exemplar hand sanitizing composition includes: effective amounts of an alcohol for disinfecting and effective amounts of CBD and/or CBG for disinfecting and serving as inflammation reducing agent. The present teachings recognize that the effective amounts of the alcohol used for sanitizing hands undesirably cause skin inflammation and the presence of CBD and/or CBG reduces this undesirable outcome. In the present compositions, effective amounts of alcohol ranges from about 60% (by volume) to about 95% (by volume), effective amounts of CBD ranges from about 0.1% (by volume) to about 0.4% (by volume); and effective amounts of CBG ranges from about 0.01% (by volume) to about 0.1% (by volume). The present sanitizing compositions have a viscosity that ranges from about 1.05 cp to about 1.2 cp.

To the extent water is used as a carrier fluid in the present compositions, the water concentration ranges from about 25% (by volume) to about 35% (by volume). The present compositions, preferably, comprise a thickening agent, such as glycerin and/or aloe. If used in the present compositions, the glycerin is present in a concentration that ranges from about 1% (by volume) to about 2% (by volume), and aloe is present in a concentration that ranges from about 1% (by volume) to about 5% (by volume).

The systems and methods of operation and effective compositions obtained from the present teachings and arrangements, however, together with additional objects and advantages thereof, will be best understood from the following descriptions of specific embodiments when read in connection with the accompanying figures.

### BRIEF DESCRIPTION OF THE DRAWINGS

FIG. 1 shows a schematic diagram of a sanitizing composition producing system, according to one embodiment of the present arrangements, for producing sanitizing compositions that effectively disinfect many different types of surfaces and if applied on skin, sanitize without causing skin inflammation or irritation.

FIG. 2A shows an exploded view of a first homogenizing subsystem, according to one embodiment of the present arrangements, including a first homogenizing blade having multiple first homogenizing teeth.

FIG. 2B shows an exploded view of a second homogenizing subsystem, according to one embodiment of the present arrangements, including a second homogenizing blade having multiple second homogenizing teeth.

FIG. 3 shows a flowchart for a sanitizing composition producing process, according to one embodiment of the present teachings, for producing sanitizing compositions.

### DETAILED DESCRIPTION OF THE DRAWINGS

In the following description, numerous specific details are set forth in order to provide a thorough understanding of the present teachings and arrangements. It will be apparent, however, to one skilled in the art that the present teachings and arrangements may be practiced without limitation to some or all these specific details. By way of example, the present systems and methods are described in terms of batch mode of operation, but continuous mode of operation is equally applicable to implement the present teachings. In other instances, well-known process steps have not been described in detail in order to not unnecessarily obscure the present teachings and arrangements.

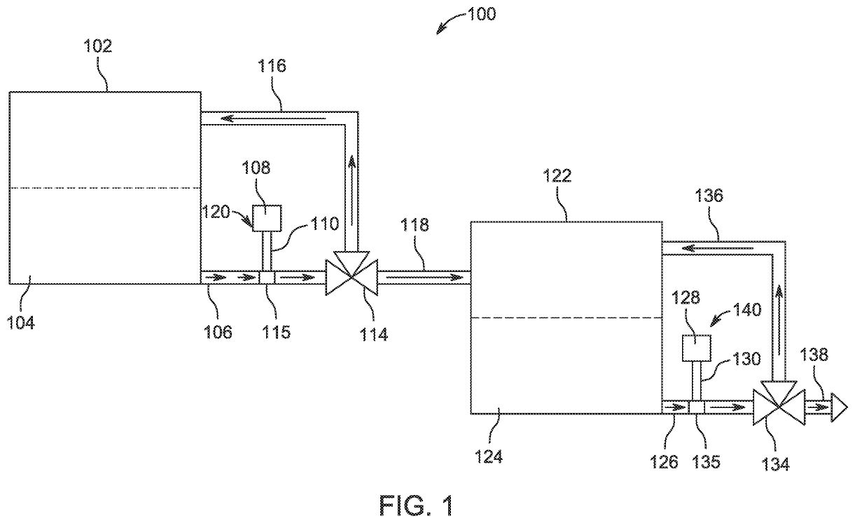

FIG. 1 shows a sanitizing composition producing system 100, according to one embodiment of the present arrangements, for producing sanitizing compositions. Sanitizing composition producing system 100 is generally a multi-stage reactor assembly and, preferably a two-stage reactor assembly. To this end, sanitizing composition producing system 100, preferably, includes two reactors disposed adjacent to their respective downstream homogenizing subassemblies, i.e., a first reactor 102 disposed downstream from a first homogenizing subsystem 120 and a second reactor 122 disposed downstream from a second homogenizing subsystem 140. The homogenizing subsystems serve to homogenize the fluid contents inside their respective reactors so that by repeated reaction, homogenization and recirculation of such fluid contents, an effective sanitizing composition of the present teachings is produced.

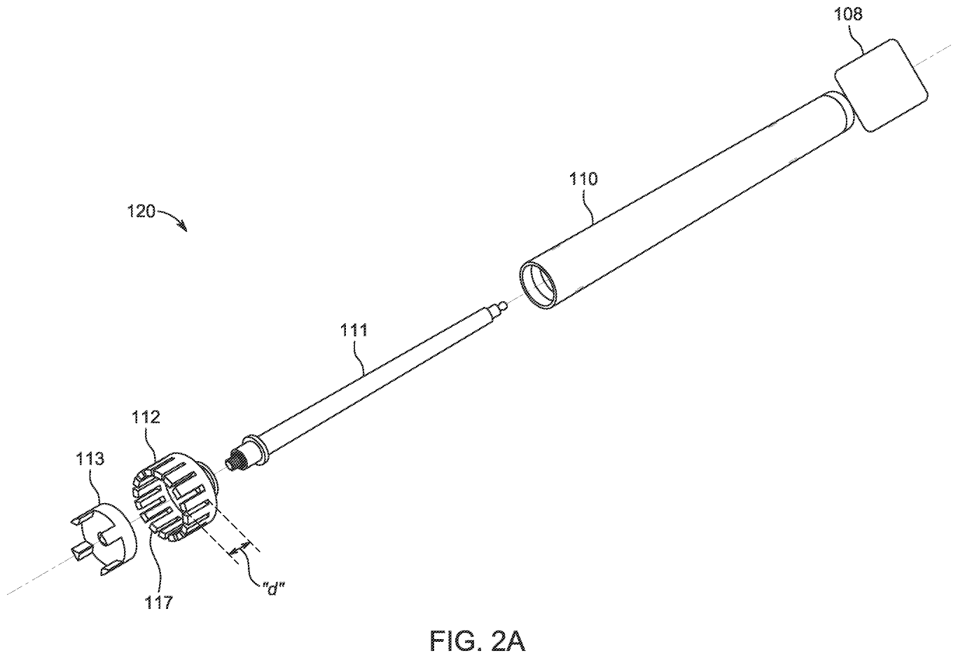

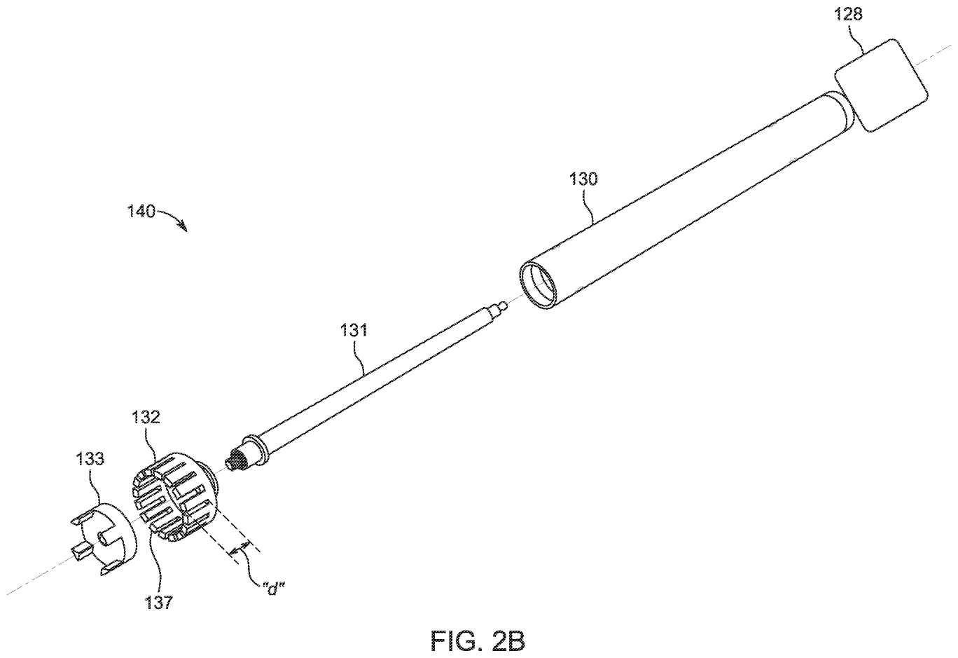

Inside first reactor 102, fluid 104 represents an intermediate sanitizing composition that is conveyed downstream from a first outlet line 106 to first homogenizing subsystem 120 (shown in greater detail in FIG. 2A). First homogenizing subsystem 120 includes a first motor 108 rotationally driving a first axle 111 that longitudinally extends inside a first shaft 110, which preferably includes a bushing to receive a first homogenizing blade 112. Further, a first rotor 113 is received within a central region of a first homogenizing blade, defining a first gap there-between. In this configuration, first axle 111 connects to a first rotor 113 such that during an operative state of first homogenizing subsystem 120, a rotating action of first axle 111 causes first rotor 113 to rotate. In this operative state of first homogenizing subsystem 120, the rotation action of first rotor 113 occurs relative to a first homogenizing blade 112, which is preferably not rotating. As will be explained below, fluid passing through a first rotor/homogenizing blade subassembly 115 (e.g., specifically a first gap defined by a space between first rotor 113 and first homogenizing blade 112) undergoes effective homogenization.

Intermediate sanitizing composition homogenized at first homogenizing subsystem 120 recirculates, preferably using a three-way valve 114 and first recirculation line 116, from and back to first reactor 102. To facilitate such recirculation, one or more valves (e.g., three-way valve 114), in one setting, is set to prevent fluid flow from outlet line 106 to flow into an input line 118, thereby directing flow through first recirculation line 116 and back to first reactor 102. In this setting, a recirculation loop around first reactor 102 and first homogenizing subsystem 120 is formed to effectively repeatedly subject the intermediate sanitizing composition to undergo, for a certain time duration, reaction and mixing inside first reactor 102, homogenizing inside first homogenizing subsystem 120 and recirculation using one or more valves and through first recirculation line 116, until the intermediate sanitizing composition is an alcohol and water solution of a predefined concentration. By way of example, the alcohol in the intermediate sanitizing composition may range from about 65% (by volume) to about 95% (by volume). In one embodiment of the present arrangements, to effectively react the alcohol and water mixture inside first reactor 102 at the appropriate reaction temperatures, outer walls of first reactor 102 are surrounded by a heating jacket. In certain embodiments, the repeated steps of reaction and mixing, homogenizing and recirculation are carried out for a predefined time duration that ensures that the components (e.g., alcohol and/or water) are present in the intermediate composition at the requisite concentration or one or more different types of sensors (e.g., concentration sensors) are used to ensure that the predefined component concentrations are reached so that preparation may be made to commence the next stage of reaction.

After a predefined time duration of reaction, homogenization and/or recirculation, and/or after reaching an intermediate sanitizing composition having desired concentration of chemical components present therein (e.g., alcohol and/or water), the intermediate sanitizing composition may be cooled to within a desired temperature range and then transferred through one or more valves (e.g., a three-way valve 114) and to input line 118 so that processing may commence at the second stage of reaction and mixing. Specifically, one or more valves (e.g., a three-way valve 114) is set to prevent fluid from first outlet line 106 to flow into recirculation line 116 and allowed to convey the intermediate composition to second reactor 122.

In connection with the second stage of reaction, second reactor 122, a second outlet line 126, second homogenizing subsystem 140 (shown in greater detail in FIG. 2B), which includes a second motor 128, a second shaft 130, and a second rotor/homogenizing blade subassembly 135, each of which are substantially similar to and arranged similar to their counterparts discussed with respect to first reactor 102, e.g., first outlet line 106, first homogenizing subsystem 120, first motor 108, first shaft 110, and first rotor/homogenizing blade subassembly 115. Second reactor 122 may be operating at a temperature that ranges from about 10° C. and to 30° C. and is, preferably, operating at or below room temperature. Intermediate sanitizing composition of a desired composition and sufficiently homogenized is, preferably, cooled to arrive at a “cooled-intermediate sanitizing composition” and then conveyed through valve 114 and input line 118 to second reactor 122.

At the relatively low prevailing temperatures inside second reactor 122 and using second homogenizing subsystem 140, the first homogenized composition of alcohol and water is repeatedly reacted, mixed, homogenized and/or recirculated to produce a second homogenized solution 124, which is, preferably, the effective sanitizing composition of the present teachings. In other embodiments of the present arrangements, one or more additional stages of reacting, mixing, homogenizing and/or recirculation may be carried out, but the arrangement of FIG. 1 represents a preferred embodiment of the present arrangements. If additional reaction stages are used, then another set of one or more valves (e.g., three-way valve 134) along with a two-stage homogenized sanitizing composition outlet 138 is used to convey a two-stage homogenized sanitizing composition for further reaction, homogenization and/or recirculation to a downstream reaction stage for further processing. If additional reaction stages are not used, then another set of one or more valves (e.g., three-way valve 134) and two-stage homogenized sanitizing composition outlet 138 are used to dispense the sanitizing composition of the present teachings.

In certain embodiments of the present arrangements, one or more inlets into first reactor 102 and/or second reactor 122 are provided to supply any at least member chosen from a group comprising thickening agent, additive and fragrance. Regardless of the type of contents received inside first reactor 102 and/or second reactor 122, these contents undergo homogenization to produce an effective sanitizing composition.

FIG. 2A shows in greater detail first homogenizing subsystem 120, which includes first motor 108 for rotationally driving a first axle 111 that is coupled to first rotor 113. In an assembled configuration of first homogenizing subsystem 120, first axle 111 longitudinally extends inside first shaft 110, which is preferably fitted with a bushing that couples to a first homogenizing blade 112. During an operative state of first homogenizing subsystem 120, a rotational action of first axle 111 cause first rotor 113 to rotate relative to homogenizing blade 112, which is preferably not rotating. In this operative state of the assembled configuration shown in FIG. 2A, the intermediate sanitizing composition flows through a first gap defined between first homogenizing blade 112 and first rotor 113, and thereby undergoes homogenization. Specifically, first homogenizing blade 112 have multiple first homogenizing teeth 117 that, in an operative state, facilitate homogenization of the intermediate sanitizing composition or any fluid passing through the first gap.

As shown in FIG. 2A, first homogenizing teeth 117 protrude from a first teeth base portion, which is denoted by “d” and facilitate the homogenization of the intermediate sanitizing composition having a viscosity that ranges from about 1.05 cp to about 1.2 cp. In one embodiment, “d,” in first homogenizing blade 112, ranges from about 0.5 mm and about 1.2 mm to achieve the effective homogenization of the intermediate sanitizing composition.

FIG. 2B, similar to FIG. 2A, shows in greater detail second homogenizing subsystem 140, which, in an assembled configuration, includes second motor 128 rotationally driving a second axle 130 that is coupled to second rotor 133. Further, second axle 131 longitudinally extends inside second shaft 130, which is, preferably, fitted with a bushing that couples to a second homogenizing blade 132.

During an operative state of second homogenizing subsystem 140, motor 128 causes rotational motion of first axle 131, which in turn rotationally displaces first rotor 133 relative to second homogenizing blade 132, which is preferably not rotating. In this operative state, sanitizing composition 124 shown in FIG. 1 flows through a second gap defined between second homogenizing blade 132 and second rotor 133 undergoes homogenization. Specifically, second homogenizing blade 132 having multiple second homogenizing teeth 137 that, in an operative state, facilitate homogenization of sanitizing composition 124 passing through the second gap.

As shown in FIG. 2B, second homogenizing teeth 137 protrude from a second teeth base portion, which is denoted by “d.” In one embodiment, “d” (e.g., inside second homogenizing subsystem 140) is sufficiently large to effectively homogenize a mixture of alcohol and water solution in the second reaction stage (e.g., inside second reactor 122) having a viscosity that ranges from about 1.05 cp to about 1.2 cp. In preferred embodiments, “d” ranges from about 1.25 mm and about 3.5 mm to achieve the effective homogenization of sanitizing composition 124.

First axle 111 and/or second axle 131 are, preferably, specialized dispersing axles, which, in one operative state, have a rotational speed ranging from about 10,000 rotations per minute (“rpms”) to 30,000 rpms to effectively disperse relatively viscous compositions of the intermediate sanitizing compositions and/or sanitizing compositions of the present teachings.

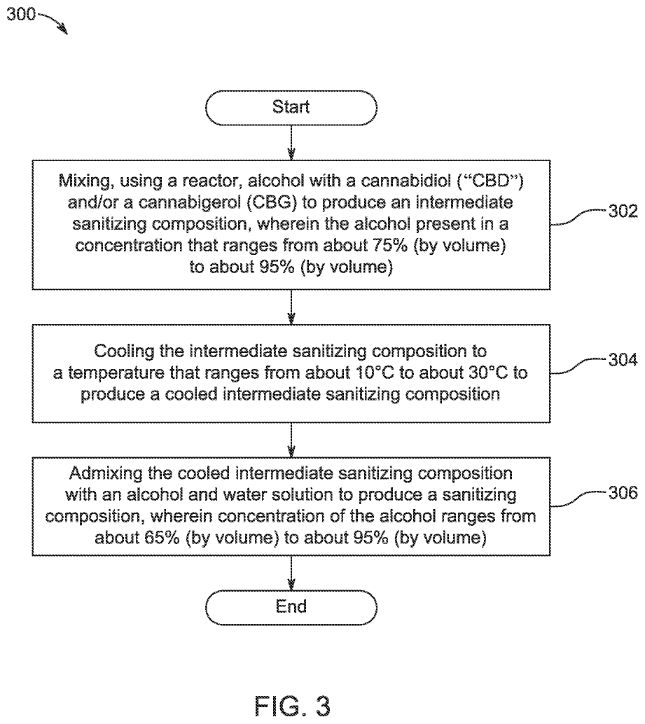

The present teachings also provide methods for producing hand-sanitizing compositions. Certain of these methods need not be carried out using structural details described in connection with FIG. 1 or otherwise. FIG. 3 shows a flowchart for a method 300, according to one preferred embodiment of the present teachings, for producing the present sanitizing compositions.

Method 300 begins with a step 302, which involves mixing, using a reactor at a reaction temperature that is between about 50° C. and about 70° C., alcohol with a cannabidiol (“CBD”) and/or a cannabigerol (CBG) to produce an intermediate sanitizing composition, in which the alcohol is present in a concentration that ranges from about 75% (by volume) to about 95% (by volume). In preferred embodiments of step 302, in one part by volume of alcohol, each of CBD or CBG is also present in about one part by volume. In other words, the ratio of alcohol to each CBD or CBG is, preferably, approximately 1:1 when mixing alcohol with either of the two. In those instances where a mixture of CBD and CBG is used to form the intermediate sanitizing composition, for one part by volume of alcohol, a mixture of CBD and CBG is present in about one part by volume (i.e., ratio of alcohol to a mixture CBD and CBG is, preferably, approximately 1:1).

Step 302 may include receiving different types of raw materials, such as alcohol, CBD and/or CBG, or may include additives, such as thickening agents or fragrances. Each of the raw material and/or thickening agents are preferably stored in their respective reservoirs and are conveyed through their respective input lines to first reactor 102 and/or second reactor 122 as is deemed desirable.

Next, a step 304 including cooling the intermediate sanitizing composition to a temperature that ranges from about 10° C. to about 30° C. to produce a “cooled intermediate sanitizing composition.” In a preferred embodiment of step 304, the cooling step includes cooling the intermediate sanitizing composition to a relatively lower temperature, which is cooler than the reaction temperature of alcohol and water solution at the first reaction stage (e.g., inside first reactor 102).

In one implementation of the present teachings, method 300 may include a conveying step that includes batchwise conveying the cooled intermediate sanitizing composition from first reactor 102 to second reactor 122 at an appropriate time after lapse of a predefined time duration of the intermediate sanitizing composition undergoing homogenization and recirculation in the first reaction stage or when one or more predefined conditions (e.g., temperature) for the cooled intermediate sanitizing composition are achieved. One or more additives, such as lavender essential oil, hemp essential oil, terpenes and hydrosols may be introduced into first reactor 102 and/or into second reactor 122 (i.e., introduced when the cooled intermediate sanitizing composition is present inside second reactor 122).

Then, a step 306 requires admixing the cooled intermediate sanitizing composition with an alcohol water solution to produce the sanitizing composition, wherein the concentration of the alcohol ranges from about 65% to about 95% (by volume).

Although the present teachings do not limit the different steps involved in producing a sanitizing composition to be carried out in any particular order, preferred embodiments of the present teachings recognize that the lipophilic nature of CBD or CBG desire a specific order in which the steps should be carried out. According to these preferred embodiments, prior to cooling step 304, methods 300 involve preparing the intermediate sanitizing composition for thickening by cooling the intermediate sanitizing composition to a temperature that ranges from about 30° C. and to 50° C. to produce a thickened intermediate sanitizing composition. I n more preferred embodiments, the preparing step of the present teachings is carried out by cooling the intermediate sanitizing composition to room temperature or even lower temperatures to achieve the requisite thickening.

One embodiment of the preparing step includes introducing into a first reactor (e.g., first reactor 102 of FIG. 1) a thickening agent to produce a thickened intermediate sanitizing composition. By way of example, the thickening agent is glycerin and/or an aloe-based compound. The preparing step to produce the thickened intermediate sanitizing composition may include introducing into the reactor one or more different types of fragrances. The fragrance may be an essential oil, such as a hemp-based essential oil and/or a lavender-based essential oil.

In preferred embodiments, method 300 further comprises recirculating, using a first recirculation line (e.g., first recirculating line 116 of FIG. 1), an intermediate sanitizing composition (e.g., intermediate sanitizing composition 104 of FIG. 1) from a first reactor (e.g., first reactor 102 of FIG. 1) back to the first reactor. This recirculating step is performed prior to step 306 of admixing the cooled intermediate sanitizing composition with an alcohol water solution to produce the sanitizing composition.

In this preferred embodiment, method 300 also comprises homogenizing the intermediate sanitizing composition using a first homogenizing blade (e.g., first homogenizing blade 112 of FIG. 2A) having a first homogenizing teeth (e.g., first homogenizing teeth 117 of FIG. 2A) that protrude, from a first teeth base portion, a distance (e.g., protruding distance “d” shown in FIG. 2A) ranging from about 0.5 mm to about 1.2 mm. In this configuration, the first homogenizing blade is disposed on the first recirculating line as shown in FIG. 1 and homogenization is carried out by, preferably, passing the intermediate sanitizing composition through a first gap defined between a first rotor and a first homogenizing blade. In this manner, method 300, preferably includes repeated mixing step 302, i.e., reacting, homogenizing and/or recirculating.

In preferred embodiments, method 300 similarly further comprises recirculating, using a second recirculation line (e.g., second recirculating line 136 of FIG. 1), a sanitizing composition (e.g., sanitizing composition 124 of FIG. 1) from a second reactor (e.g., second reactor 122 of FIG. 1) back to the second reactor. In this preferred embodiment, method 300 also comprises homogenizing the sanitizing composition using a second homogenizing blade (e.g., second homogenizing blade 132 of FIG. 2B) having a second homogenizing teeth (e.g., second homogenizing teeth 137 of FIG. 2B) that protrude, from a second teeth base portion, a distance (e.g., protruding distance “d” shown in FIG. 2B) ranging from about 1.25 mm to about 3.5 mm. In this configuration, the second homogenizing blade is disposed on the second recirculating line as shown in FIG. 1 and homogenization is carried out by, preferably, passing the sanitizing composition through a second gap defined between a second rotor and a second homogenizing blade. In this manner, method 300, preferably includes repeated admixing step 306, i.e., reacting, homogenizing and/or recirculating in the second stage of processing.

The present teachings also offer effective sanitizing compositions to sanitize skin (e.g., hand) and various surfaces. In one preferred embodiment of the present teachings, present sanitizing compositions comprise effective amounts of an alcohol for disinfecting and effective amounts of CBD and/or CBG for disinfecting and serving as inflammation reducing agent. In the event the sanitizing composition is used on skin, effective amounts of alcohol that sanitize the skin undesirably causes skin inflammation. In these preferred embodiments, effective amounts of alcohol ranges from about 60% (by volume) to about 95% (by volume) and effective amounts of CBD ranges from about 0.1% (by volume) to about 0.4% (by volume), and effective amounts of CBG ranges from about 0.01% (by volume) to about 0.1% (by volume). The present sanitizing compositions have a viscosity that ranges from about 1.05 cp to about 1.2 cp.

The present sanitizing composition comprises water acting as a carrier fluid and present, preferably, in a concentration that ranges from about 25% (by volume) to about 35% (by volume). In this preferred embodiment, the present sanitizing composition further comprises a thickening agent that includes glycerin and/or aloe. Preferably, glycerin is present in a concentration that ranges from about 1% (by volume) to about 2% (by volume), and aloe is present in a concentration that ranges from about 1% (by volume) to about 5% (by volume).

The present teachings also contemplate the possibility that hand-sanitizing compositions of the present teachings may further include additives (e.g., essential oils) which impart fragrance upon application of the sanitizing compositions. By way of example, such additives include one or more compounds chosen from a group comprising lavender essential oil, hemp essential oil, terpenes and hydrosols. Further, the additives may be present in a concentration that ranges from about 0.01% (by volume) to about 5% (by volume).

In the following description, numerous specific details are set forth in order to provide a thorough understanding of the present teachings and arrangements. It will be apparent, however, to one skilled in the art that the present teachings and arrangements may be practiced without limitation to some or all of these specific details. In other instances, well-known process steps have not been described in detail in order to not unnecessarily obscure the present teachings and arrangements.

What is claimed is:

1. An apparatus for producing a sanitizing composition, the apparatus comprising:
one or more first reactors for heating alcohol with a cannabidiol (“CBD”) and/or a cannabigerol (CBG) to produce an intermediate sanitizing composition, wherein said alcohol is at a temperature that is between about 50° C. and about 70° C.; and
a first homogenizing subsystem including a first homogenizing blade for mixing alcohol with said intermediate sanitizing composition to produce a first homogenized composition.

2. The apparatus of claim 1 for producing a sanitizing composition, further comprising a first recirculation line that recirculates an output of said one or more of said first reactors back to one or more of said first reactors, wherein said first recirculation line has disposed thereon or proximate thereto a first homogenizing subsystem for homogenizing said output from said one or more of said first reactors.

3. The apparatus of claim 2 for producing a sanitizing composition, wherein said first homogenizing subsystem is located downstream from one or more of said first reactors, wherein said first homogenizing subsystem comprises a first motor, a first rotor, a first shaft, a first axle and a first homogenizing blade, wherein, in an assembled state of said first homogenizing subsystem, said first motor is coupled to drive said first axle, which is coupled to said first rotor, wherein said first axle longitudinally extends inside said first shaft, which receives said first homogenizing blade, and wherein said first rotor is received within a central region of said first homogenizing blade, defining a first gap therebetween to allow for fluid flow between said first rotor and said first homogenizing blade.

4. The apparatus of claim 3 for producing a sanitizing composition, wherein said first blade has first homogenizing teeth that protrude, from a first teeth base portion, a distance ranging from about 0.5 mm to about 1.2 mm to effectively homogenize said intermediate sanitizing composition having a high viscosity ranging from about 1.05 centipoise (cp) to about 1.2 cp.

5. The apparatus of claim 3 for producing a sanitizing composition, further comprising one or more valves designed to direct fluid inside one or more of said first reactors to said first recirculation line or to a second reaction stage.

6. The apparatus of claim 1 for producing a sanitizing composition, further comprising:
one or more second reactors for reacting and mixing said intermediate sanitizing composition and an alcohol and water solution to produce a two-stage homogenized sanitizing composition or said sanitizing composition; and
second homogenizing subsystem for homogenizing said two-stage homogenized sanitizing composition or said sanitizing composition.

7. The apparatus of claim 6 for producing a sanitizing composition, further comprising a second recirculation line that recirculates an output from said one or more of said second reactors back to one or more of said second reactors, wherein said second recirculation line has disposed thereon or proximate thereto said second homogenizing subsystem for homogenizing said output from said one or more of said second reactors.

8. The apparatus of claim 7 for producing a sanitizing composition, wherein said second homogenizing subsystem is located downstream from one or more of said second reactors, wherein said second homogenizing subsystem comprises a second motor, a second rotor, a second shaft, a second axle and said second homogenizing blade, wherein, in an assembled state of said second homogenizing subsystem, said second motor is coupled to drive said second axle, which is coupled to said second rotor, wherein said second axle longitudinally extends inside said second shaft, which receives said second homogenizing blade, and wherein said second rotor is received within a central region of said second homogenizing blade, defining a second gap therebetween to allow for fluid flow between said second rotor and said second homogenizing blade.

9. The apparatus of claim 8 for producing a sanitizing composition, wherein said second homogenizing blade has second homogenizing teeth that protrude, from a second teeth base portion, a distance ranging from about 1.25 mm to about 3.5 mm.

10. A method for producing a sanitizing composition, said method comprising:
mixing, using a first reactor, alcohol with a cannabidiol (“CBD”) and/or a cannabigerol (CBG) to produce an intermediate sanitizing composition, wherein said alcohol is at a temperature that is between about 50° C. and about 70° C., and wherein said alcohol present in a concentration that ranges from about 75% (by volume) to about 95% (by volume);
cooling said intermediate sanitizing composition to a temperature that ranges from about 10° C. to about 30° C. to produce a cooled intermediate sanitizing composition; and
admixing, using a second reactor, said cooled intermediate sanitizing composition with an alcohol and water solution to produce said sanitizing composition, wherein concentration of said alcohol in said ranges from about 65% (by volume) to about 95% (by volume).

11. The method of claim 10 for producing a sanitizing composition, wherein in said mixing to produce an intermediate sanitizing composition, for one part by volume of said alcohol, each of CBD or CBG are present in about one part by volume.

12. The method of claim 10 for producing a sanitizing composition, wherein in said mixing to produce an intermediate sanitizing composition, for one part by volume of said alcohol, a mixture of CBD and CBG is present in about one part by volume.

13. The method of claim 10 for producing a sanitizing composition, wherein prior to cooling said intermediate sanitizing composition, preparing said intermediate sanitizing composition for thickening by cooling said intermediate sanitizing composition to a temperature that ranges from about 30° C. and to 50° C. to produce a thickened intermediate sanitizing composition.

14. The method of claim 13 for producing a sanitizing composition, wherein in said preparing to produce said thickened intermediate sanitizing composition, introducing into said reactor a thickening agent.

15. The method of claim 14 for producing a sanitizing composition, wherein said thickening agent is glycerin and/or an aloe-based compound.

16. The method of claim 10 for producing a sanitizing composition, wherein said mixing further comprises:
recirculating, using a first recirculation line, said intermediate sanitizing composition from said first reactor back to said first reactor, and
homogenizing said intermediate sanitizing composition using a first homogenizing blade having a first homogenizing teeth that protrude, from a first teeth base portion, a distance ranging from about 0.5 mm to about 1.2 mm, wherein said first homogenizing blade is disposed on said first recirculating line.

17. The method of claim 10 for producing a sanitizing composition, wherein said admixing further comprises:
recirculating, using a second recirculation line, said sanitizing composition from said second reactor back to said second reactor, and
homogenizing said sanitizing composition using a second homogenizing blade having a second homogenizing teeth that protrude, from a second teeth base portion, a distance ranging from about 1.25 mm to about 3.5 mm, wherein said second homogenizing blade is disposed on said second recirculating line.

18. A hand sanitizing composition comprising:
effective amounts of an alcohol for disinfecting;
effective amounts of CBD and/or CBG for disinfecting and serving as inflammation reducing agent, wherein said effective amounts of said alcohol produces skin inflammation,
wherein said effective amounts of said alcohol ranges from about 60% (by volume) to about 95% (by volume),
wherein said effective amounts of CBD ranges from about 0.1% (by volume) to about 0.4% (by volume); and
wherein said effective amounts of CBG ranges from about 0.01% (by volume) to about 0.1% (by volume).

19. The hand sanitizer composition of claim 18, further comprising water acting as a carrier fluid and in a concentration that ranges from about 25% (by volume) to about 35% (by volume).

20. The hand sanitizer composition of claim 18, further comprising a thickening agent that includes glycerin and/or aloe, wherein said glycerin is present in a concentration that ranges from about 1% (by volume) to about 2% (by volume), and wherein said aloe is present in a concentration that ranges from about 1% (by volume) to about 5% (by volume).
